(19) United States
(12) Patent Application Publication
(10) Pub. No.: US 2024/0358429 A1
(43) Pub. Date: Oct. 31, 2024

(54) TREATMENT TOOL, TREATMENT SYSTEM, AND METHOD OF OPERATING TREATMENT SYSTEM

(71) Applicant: OLYMPUS CORPORATION, Tokyo (JP)

(72) Inventors: Shinichi KITADE, Tokyo (JP); Kazuya NAKABE, Tokyo (JP); Katsushi WATANABE, Tokyo (JP); Soichi IKUMA, Tokyo (JP)

(73) Assignee: OLYMPUS CORPORATION, Tokyo (JP)

(21) Appl. No.: 18/643,584

(22) Filed: Apr. 23, 2024

Related U.S. Application Data
(60) Provisional application No. 63/461,610, filed on Apr. 25, 2023.

(51) Int. Cl. A61B 18/14; A61B 18/00

(52) U.S. Cl. CPC A61B 18/14; A61B 2018/00083; A61B 2018/00517; A61B 2018/00601; A61B 2018/00982; A61B 2018/1412; A61B 2218/007

(57) ABSTRACT

A treatment tool includes: a tubular portion including a tubular portion main body having a long tubular shape and an opening section provided in the tubular portion main body, the tubular portion being configured to suck a tissue piece from the opening section; a blade portion provided in a part of an opening edge in the opening section; and a non-blade portion provided in at least a part other than a region where the blade portion is provided in the opening edge, the non-blade portion being configured to slide the tissue piece. The blade portion includes an edge having a smallest sectional area in a cross section of the blade portion taken along a plane including a central axis of the tubular portion main body, and only the edge is located on an imaginary plane obtained by extending an opening surface of the opening section.

### CROSS-REFERENCE TO RELATED APPLICATION

This application is based on and claims priority under 35 U.S.C. § 119 to U.S. Provisional Application No. 63/461,610, filed Apr. 25, 2023, the entire contents of which are incorporated herein by reference.

### BACKGROUND

### 1. Technical Field

The present disclosure relates to a treatment tool, a treatment system, and a method of operating the treatment system.

### 2. Related Art

In prostate enucleation surgery in which an enucleated prostate tissue (hereinafter described as tissue piece) is extracted in holmium laser nucleation of the prostate (HoLEP) that is a surgical treatment for benign prostatic hyperplasia (BPH), a treatment tool called morcellator has been used (see, for example, U.S. Pat. No. 6,024,751).

The treatment tool described in U.S. Pat. No. 6,024,751 includes an insertion section including an inner tube in which a first opening section is provided in a side wall and an outer tube in which the inner tube is inserted through the inside and in which a second opening section is provided in a side wall. Then, in the treatment tool, a tissue piece nucleated by laser irradiation in the holmium laser enucleation of the prostate is sequentially cut as explained below and sucked through the inside of the inner tube to thereby be discharged from the inside of the bladder to the outside of the body.

Specifically, the first and second opening sections are superimposed by relatively sliding the inner tube and the outer tube along the longitudinal axis. Since the tissue piece is sucked through the inner tube, a part of the tissue piece is drawn into the inside of the inner tube from an aperture in which the first and second opening sections overlap each other. Next, the inner tube and the outer tube are relatively slid along the longitudinal axis to narrow the area of the aperture in which the first and second opening sections overlap each other. As a result, the part of the tissue piece drawn into the inside of the inner tube from the aperture is cut by an edge portion of the opening functioning as a cutting blade. The cut part of the tissue piece is discharged to the outside of the body through the inside of the inner tube.

The operation explained above is repeated, whereby the tissue piece is sequentially cut and discharged from the inside of the bladder to the outside of the body.

### SUMMARY

In some embodiments, a treatment tool includes: a tubular portion including a tubular portion main body having a long tubular shape and an opening section provided in the tubular portion main body, the tubular portion being configured to suck a tissue piece from the opening section; a blade portion provided in a part of an opening edge in the opening section; and a non-blade portion provided in at least a part other than a region where the blade portion is provided in the opening edge, the non-blade portion being configured to slide the tissue piece. The blade portion includes an edge having a smallest sectional area in a cross section of the blade portion taken along a plane including a central axis of the tubular portion main body, and only the edge is located on an imaginary plane obtained by extending an opening surface of the opening section.

A treatment system includes: a treatment tool configured to be inserted into a subject; and a suction device configured to suck a tissue piece through the treatment tool, the treatment tool including: a tubular portion including a tubular portion main body having a long tubular shape and an opening section provided in the tubular portion main body, the tubular portion being configured to suck the tissue piece from the opening section; a blade portion provided in a part of an opening edge in the opening section; and a non-blade portion provided in at least a part other than a region where the blade portion is provided in the opening edge, the non-blade portion being configured to slide the tissue piece. The blade portion includes an edge having a smallest sectional area in a cross section of the blade portion taken along a plane including a central axis of the tubular portion main body, and only the edge is located on an imaginary plane obtained by extending an opening surface of the opening section.

Provided is a method of operating a treatment system including a treatment tool configured to be inserted into a subject and a suction device configured to suck a tissue piece through the treatment tool, the treatment tool including a tubular portion including a tubular portion main body having a long tubular shape and an opening section provided in the tubular portion main body, the tubular portion being configured to suck the tissue piece from the opening section, a blade portion provided in a part of an opening edge in the opening section, and a non-blade portion provided in at least a part other than a region where the blade portion is provided in the opening edge, the non-blade portion being configured to slide the tissue piece, the blade portion includes an edge having a smallest sectional area in a cross section of the blade portion taken along a plane including a central axis of the tubular portion main body, and only the edge is located on an imaginary plane obtained by extending an opening surface of the opening section. The method includes: scraping the tissue piece with the blade portion by applying high-frequency power to the blade portion while attracting the tissue piece to the opening section of the tubular portion main body with suction of the suction device; and collecting the scraped tissue piece from the tubular portion main body with the suction.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment.

FIG. 2 is a diagram for explaining a configuration of an insertion section.

FIG. 3 is a diagram for explaining the configuration of the insertion section.

FIGS. 4A to 4C are diagrams for explaining a treatment method.

FIG. 5 is a diagram for explaining the treatment method.

FIG. 6 is a diagram for explaining an effect of the embodiment.

FIG. 7 is a diagram for explaining a modification 1 of the embodiment.

FIG. 8 is a diagram for explaining a modification 2 of the embodiment.

FIG. 9 is a diagram for explaining a modification 3 of the embodiment.

FIG. 10 is a diagram for explaining a modification 4 of the embodiment.

FIGS. 11A and 11B are diagrams for explaining a modification 5 of the embodiment.

FIG. 12 is a diagram for explaining a modification 5 of the embodiment.

FIG. 13 is a diagram for explaining a modification 6 of the embodiment.

FIG. 14 is a diagram for explaining the modification 6 of the embodiment.

### DETAILED DESCRIPTION

A mode for carrying out the disclosure (an embodiment) is explained below with reference to the drawings. Note that the disclosure is not limited by the embodiment explained below. Further, in the description of the drawings, the same portions are denoted by the same reference numerals and signs.

### Configuration of a Treatment System

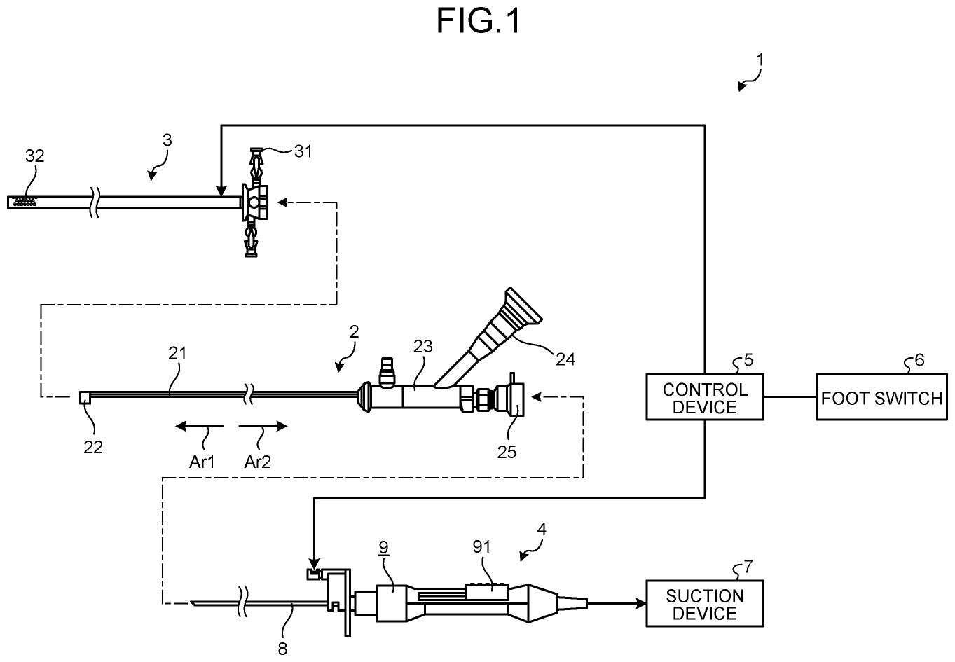

FIG. 1 is a diagram illustrating a treatment system 1 according to an embodiment. Note that, in FIG. 1, an arrow indicating an insertion direction is indicated by an alternate long and short dash line and an arrow indicating control of components or suction is indicated by a solid line.

The treatment system 1 is a nucleate fragment collection system used in prostate enucleation surgery. For example, the treatment system 1 is used in prostate enucleation surgery in which an enucleated prostate tissue (hereinafter described as tissue piece) is extracted in holmium laser nucleation of the prostate (HoLEP) or the like that is a surgical treatment for benign prostate hyperplasia (BPH). As illustrated in FIG. 1, the treatment system 1 includes an endoscope 2, an outer sheath 3, a treatment tool 4, a control device 5, a foot switch 6, and a suction device 7.

The endoscope 2 is configured by a rigid endoscope and is used to observe the inside of the bladder of a patient who is a subject. As illustrated in FIG. 1, the endoscope 2 includes an endoscope main body 21, a distal end portion 22, a treatment tool insertion section 23, and an eyepiece section 24.

The endoscope main body 21 is at least partially made of an electrically conductive material and has a linearly extending long shape. Then, an optical system (not illustrated) that is configured using one or a plurality of lenses and collects light from the subject is provided on the inside of the endoscope main body 21.

Note that, in the following explanation, one side (the left side in FIG. 1) of the longitudinal axis in the endoscope main body 21 is described as distal end side Ar1 (FIG. 1) and the other side (the right side in FIG. 1) is described as proximal end side Ar2 (FIG. 1).

The distal end portion 22 is at least partially made of an electrically conductive material, is provided on the outer peripheral surface of the end portion on the distal end side Ar1 of the endoscope main body 21, and has a columnar shape extending in a direction along the longitudinal axis in a state of being eccentric from the central axis of the endoscope main body 21. Although not specifically illustrated, the distal end portion 22 has a first through-hole having a circular shape in cross section penetrating the distal end portion 22 from the proximal end to the distal end along the central axis of the cylinder.

The treatment tool insertion section 23 is provided at the end portion on the proximal end side Ar2 of the endoscope main body 21 and has a long shape extending along the longitudinal axis of the endoscope main body 21. Although not specifically illustrated, the treatment tool insertion section 23 is provided with a second through-hole that penetrates the treatment tool insertion section 23 from the proximal end to the distal end along the longitudinal axis of the treatment tool insertion section 23, is coaxial with the first through-hole provided in the distal end portion 22, and has a circular shape in cross section having substantially the same inner diameter dimension as the inner diameter dimension of the first through-hole.

The eyepiece section 24 is a portion that is connected to the proximal end of the endoscope main body 21 through the treatment tool insertion section 23 and to which a camera head (not illustrated) is connected. That is, a subject image condensed by the endoscope main body 21 and having passed through the eyepiece section 24 is captured by the camera head. Then, an endoscopic image obtained by the imaging of the camera head is displayed on a display device (not illustrated).

The outer sheath 3 is at least partially made of an electrically conductive material and has a cylindrical shape having an inner diameter dimension slightly larger than the outer diameter dimension of the distal end portion 22. The distal end portion 22 and the endoscope main body 21 are inserted through the inside of the outer sheath 3 as indicated by an arrow of an alternate long and short dash line in FIG. 1. Accordingly, an opening portion on the distal end side Ar1 in the outer sheath 3 is closed by the distal end portion 22. The outer sheath 3 is electrically connected to the endoscope main body 21 and the distal end portion 22. On the other hand, an opening portion on the proximal end side Ar2 in the outer sheath 3 is closed by the treatment tool insertion section 23.

A tube (not illustrated) is connected to the end portion on the proximal end side Ar2 in the outer sheath 3. A water feeding port 31 (FIG. 1) for supplying physiological saline to the inside of the outer sheath 3 is provided.

A hole for water feeding 32 (FIG. 1) communicating between the inside and the outside of the outer sheath 3 is provided in a side wall of the end portion on the distal end side Ar1 in the outer sheath 3.

That is, the physiological saline supplied to the inside of the outer sheath 3 through the water feeding port 31 flows out to the outside of the outer sheath 3 through the hole for water feeding 32.

The treatment tool 4 is a so-called morcellator that sucks a tissue piece nucleated in enucleation of the prostate and discharges the tissue piece from the inside of the bladder to the outside of the body. As illustrated in FIG. 1, the treatment tool 4 includes an insertion section 8 and a housing 9.

The insertion section 8 is equivalent to a tubular portion. The insertion section 8 is formed in a linearly extending long shape and has an outer diameter dimension slightly smaller than the inner diameter dimensions of the first and second through-holes explained above. Then, the insertion section 8 is inserted through the first and second through-holes explained above from a proximal end portion 25 of the treatment tool insertion section 23 as indicated by an arrow of an alternate long and short dash line in FIG. 1. Accordingly, the distal end portion of the insertion section 8 is projected to the distal end side Ar1 from the distal end (the distal end portion 22) of the outer sheath 3 inserted into the inside of the bladder.

Note that a detailed configuration of the insertion section 8 is explained below.

The housing 9 is a portion that is provided at an end portion on the proximal end side Ar2 of the insertion section 8 and is gripped by an operator such as a surgeon.

The housing 9 is provided with an operating unit 91 that receives each of suction start operation and suction end operation that are user operations by the operator such as the surgeon. The operating unit 91 is provided to be movable along the longitudinal axis of the insertion section 8. Here, the suction start operation is, for example, operation of moving the operating unit 91 to the distal end side Ar1. When the suction start operation has been performed, suction of a tissue piece is started. On the other hand, the suction end operation is, for example, operation of moving the operating unit 91 to the proximal end side Ar2. When the suction end operation has been performed, the suction of the tissue piece ends.

Note that the suction start operation and the suction end operation are not limited to the operations explained above and may be operations using a push-button switch or operations using a lever.

The housing 9 is provided with a suction conduit (not illustrated) communicating with the insertion section 8.

The control device 5 supplies high-frequency power to between the outer sheath 3 and a sheath 81 explained below configuring the insertion section 8 in response to treatment start operation that is user operation on the foot switch 6 by the operator such as the surgeon.

Note that the treatment start operation is not limited to the user operation on the foot switch 6 and may be operation using a switch operated by a hand.

The suction device 7 is a device that is configured by a tank, a pump, and the like and sucks and collects a tissue piece through the treatment tool 4 (the suction conduit on the inside of the housing 9 and the insertion section 8).

### Configuration of the Insertion Section

Next, a configuration of the insertion section 8 explained above is explained.

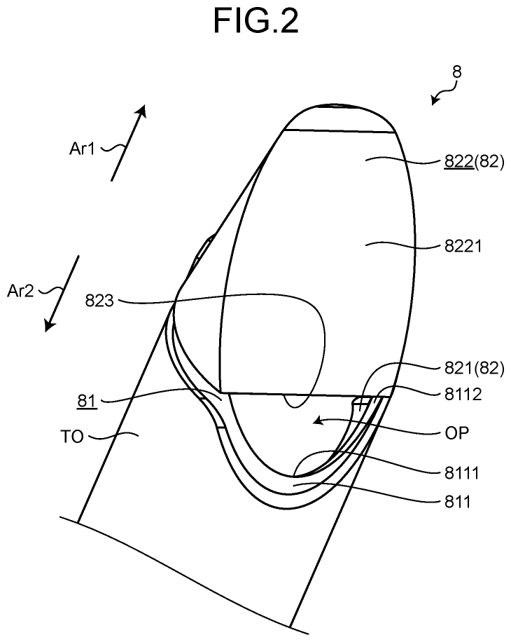

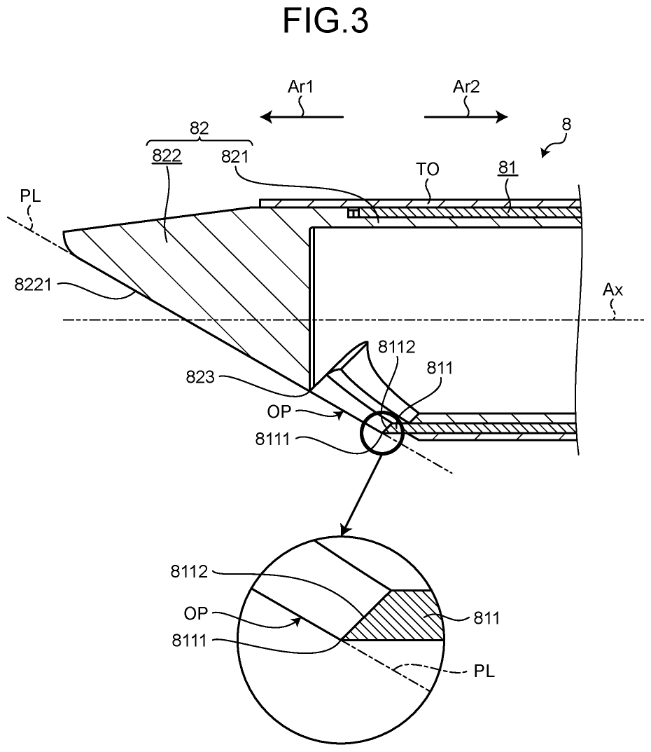

FIG. 2 and FIG. 3 are diagrams for explaining a configuration of the insertion section 8. Specifically, FIG. 2 is a perspective view illustrating the end portion on the distal end side Ar1 in the insertion section 8. FIG. 3 is a sectional view of the end portion on the distal end side Ar1 in the insertion section 8 taken along a plane extending along the central axis Ax of the insertion section 8.

As illustrated in FIG. 2 and FIG. 3, the insertion section 8 includes a sheath 81 and a non-blade portion 82.

The sheath 81 is equivalent to a tubular portion main body. The sheath 81 is a cylindrical pipe made of an electrically conductive material and is a conduit. The end portion on the proximal end side Ar2 in the sheath 81 is supported by the housing 9. The suction device 7 sucks the tissue piece through the sheath 81.

In the sheath 81, a part of an end face on the distal end side Ar1 has a shape taken along a plane PL intersecting the central axis Ax of the sheath 81 as illustrated in FIG. 3. A part of the end face has a U-shape. In the following explanation, a portion including the part of the end face is referred to as electrode 811. The electrode 811 is equivalent to a blade portion. Note that a detailed configuration of the electrode 811 is explained in “configuration of the electrode” explained below.

The non-blade portion 82 is made of rubber or the like having electrical insulation and having elasticity. A part of the non-blade portion 82 is inserted through the inside of the sheath 81 from the end portion on the distal end side Ar1 in the sheath 81 and closes a part of an opening portion on the distal end side Ar1 in the sheath 81. As illustrated in FIG. 2 and FIG. 3, the non-blade portion 82 includes an insertion section 821 and a non-blade portion main body 822.

The insertion section 821 is a portion having a cylindrical shape and inserted through the inside of the sheath 81 from the distal end side Ar1 of the sheath 81.

The non-blade portion main body 822 is integrally formed at the end portion on the distal end side Ar1 in the insertion section 821 and closes a part of an opening portion on the distal end side Ar1 in the insertion section 821. In addition, the non-blade portion main body 822 closes a part of the opening portion on the distal end side Ar1 in the sheath 81 in a state in which the non-blade portion 82 is attached to the sheath 81. More specifically, in this state, a portion on the distal end side Ar1 of the end face on the distal end side Ar1 in the sheath 81 is covered with the non-blade portion main body 822 and only the U-shaped electrode 811 on the proximal end side Ar2 is exposed.

Here, at a boundary portion between the insertion section 821 and the non-blade portion main body 822, a communication hole 823 penetrating the boundary portion from the inside toward the outside of the boundary portion is provided. A region surrounded by the electrode 811 not covered by the non-blade portion main body 822 and the communication hole 823 in the end face on the distal end side Ar1 in the sheath 81 is equivalent to an opening section OP (FIG. 2 and FIG. 3). That is, the opening section OP is formed by the sheath 81 and the non-blade portion 82. In the present embodiment, the opening section OP is located on a plane PL. More specifically, a portion located closest to the proximal end side Ar2 in an opening edge of the opening section OP is a U-shaped trough portion in the electrode 811. The distal end side Ar1 of the opening edge in the opening section OP is formed by the non-blade portion 82 and has a linear shape. That is, a positional relation between the non-blade portion 82 and the electrode 811 forming the opening edge in the opening section OP is a positional relation in which the electrode 811 is located on the proximal end side Ar2 with respect to the non-blade portion 82.

Note that the non-blade portion 82 is not limited to rubber and may be made of a material having electric insulation such as ceramic or resin.

As illustrated in FIG. 2 and FIG. 3, the outer surface of the non-blade portion main body 822 is provided with a sliding surface 8221 that, when a tissue piece is sucked into the inside of the sheath 81 from the opening section OP, slides the tissue piece toward the opening section OP. In the present embodiment, the opening surface of the opening section OP and the sliding surface 8221 form a continuous surface without a step and are provided on the same plane PL (FIG. 3). That is, the opening surface of the opening section OP intersects the central axis Ax of the sheath 81. In other words, the sliding surface 8221 is inclined from the distal end side Ar1 toward the proximal end side Ar2. A ridgeline on the proximal end side Ar2 of the sliding surface 8221 forms a part of the opening edge of the opening section OP.

As illustrated in FIG. 2 and FIG. 3, the outer surface of the sheath 81 and the outer surface of the non-blade portion 82 are covered with, in a state in which the electrode 811 and the sliding surface 8221 are exposed to the outside, an outer tube TO having electric insulation and flexibility. Here, the electrode 811 projects further toward the distal end side Ar1 with respect to the outer tube TO. The outer tube TO is equivalent to an insulating portion.

### Configuration of the Electrode

Next, a configuration of the electrode 811 is explained with reference to FIG. 2 and FIG. 3.

As illustrated in FIG. 3, the electrode 811 includes an edge 8111 and an inclined surface 8112.

The edge 8111 is a portion having the smallest sectional area in the electrode 811 when the electrode 811 is taken along a plane including the central axis Ax. The edge 8111 is a part of the end face on the distal end side Ar1 of the sheath 81 and is a portion located on the plane PL. In the electrode 811, only the edge 8111 is located on the plane PL. The plane PL corresponds to an imaginary plane. The imaginary plane is not limited to a flat surface, and may be a curved surface.

The inclined surface 8112 is located on the inner side of the sheath 81 in the electrode 811 and is connected to the edge 8111. The inclined surface 8112 is a surface extending from the edge 8111 such that the proximal end side Ar2 of the inclined surface 8112 approaches the central axis Ax of the sheath 81. In the present embodiment, the inclined surface 8112 is a surface intersecting the opening surface of the opening section OP. More specifically, the inclined surface 8112 is a surface along the normal direction of the opening surface of the opening section OP, in other words, a surface orthogonal to the opening surface of the opening section OP. That is, the inclined surface 8112 has a linearly extending shape when being taken along the plane including the central axis Ax.

Note that the inclined surface 8112 is not limited to a surface orthogonal to the opening surface of the opening section OP and may be a surface intersecting the opening surface at another angle as long as physical resistance when the tissue piece LT is sucked from the opening section OP can be reduced.

### Treatment Method

Next, a treatment method using the treatment system 1 explained above (corresponding to a method of operating the treatment system) will be described. In the following, for convenience of explanation, a method of discharging a tissue piece enucleated in enucleation of the prostate from the inside of the bladder to the outside of the body using the treatment tool 4 will be mainly described.

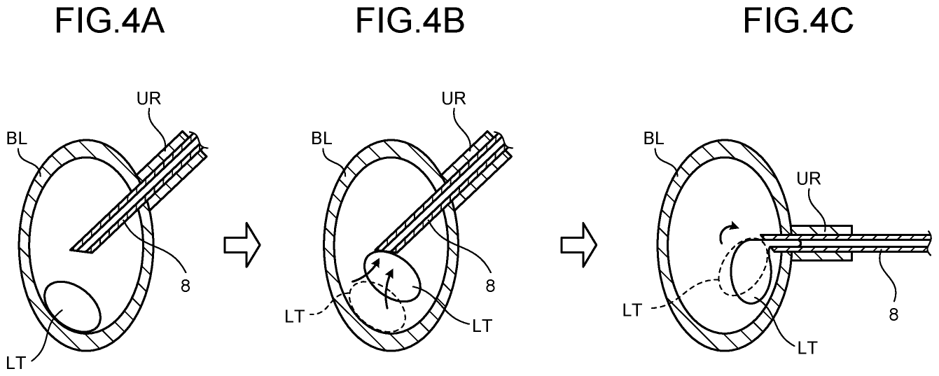

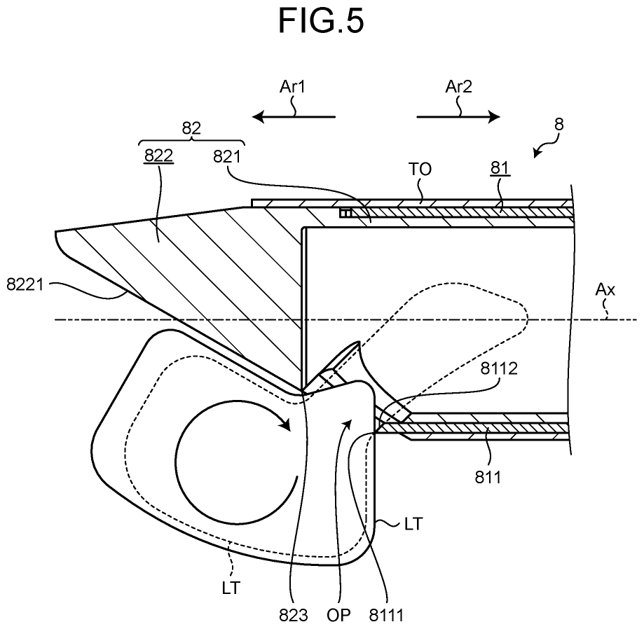

FIGS. 4A to 4C and FIG. 5 are diagrams illustrating a treatment method. FIGS. 4A to 4C illustrate a state in which the insertion section 8 is inserted into the bladder BL through the urethra UR. FIG. 5 is an enlarged view of part FIG. 4C.

In practice, the outer sheath 3 and the endoscope main body 21 are inserted into the bladder BL through the urethra UR together with the insertion section 8, but in FIGS. 4A to 4C, the outer sheath 3 and the like are not illustrated for convenience of description. The inside of the bladder BL is filled with the physiological saline flowing along the path from the water feeding port 31 to the inside of the outer sheath 3 to the hole for water feeding 32.

First, an operator such as an operator performs a suction start operation on the operating unit 91. As a result, the physiological saline is sucked from the opening section OP to the inside of the sheath 81. Then, as illustrated in FIGS. 4A and 4B, on the inside of the bladder BL, the nucleated tissue piece LT is attracted to the distal end of the insertion section 8 and held at the distal end (the sliding surface 8221 and the opening section OP) of the insertion section 8.

Next, the operator such as the surgeon performs treatment start operation on the foot switch 6. Accordingly, the control device 5 supplies high-frequency power to between the outer sheath 3 and the sheath 81. Here, the outer sheath 3 and the endoscope main body 21 and the distal end portion 22 electrically connected to the outer sheath 3 function as a collection electrode. On the other hand, the electrode 811 functions as an active electrode and scrapes off a part of the tissue piece LT on a principle explained below according to supplied high-frequency power. Then, the tissue piece LT is gradually scraped off by the electrode 811 while rotating in the direction of an arrow illustrated in FIG. 4C or FIG. 5 and a part of the scraped-off tissue piece LT is sucked into the inside of the sheath 81 from the opening section OP. That is, after a part of the tissue piece LT is sucked, the suction is continued to rotate the tissue piece LT, another part of the tissue piece LT is scraped off by the electrode 811, and the scraped-off other part of the tissue piece LT is further sucked from the opening section OP. Note that the tissue piece LT scraped off by the electrode 811 is not sucked into the inside of the sheath 81 in a finely broken state but is sucked into the inside of the sheath 81 in a connected state. A broken line in FIG. 5 illustrates a state in which the tissue piece LT scraped off by the electrode 811 is sucked into the sheath 81 in a connected state.

That is, in the treatment method according to the present embodiment, the tissue piece LT is constantly sucked into the sheath 81 from the opening section OP without changing the opening area of the opening section OP.

Note that the principle of cutting the tissue piece LT by the electrode 811 is as follows.
(1) When the supply of the high-frequency power to between the outer sheath 3 and the sheath 81 is started, the physiological saline around the electrode 811 is heated by a high-frequency current from the electrode 811.
(2) When the physiological saline around the electrode 811 reaches the boiling point, the physiological saline vaporizes and air bubbles are generated. Further, when the supply of the high-frequency power is continued, the air bubbles increase.
(3) The air bubbles cover the periphery of the electrode 811, whereby a high resistance state occurs between the entire electrode 811 and the physiological saline and spark discharge occurs. Then, air bubbles are further generated by the heat of the spark discharge and the spark discharge continues.
(4) When the entire electrode 811 is brought into contact with the tissue piece LT in a state of discharging sparks, the tissue piece LT is heated by the heat of the spark discharge, steam explosion occurs, and the tissue piece LT is scraped.

As explained above, the spark discharge explained above is important for cutting the tissue piece LT with the electrode 811.

According to the present embodiment explained above, the following effects are achieved.

In the treatment tool 4 according to the present embodiment, the electrode 811 functioning as the blade portion that scrapes the tissue piece LT is provided only in a part of the opening edge of the opening section OP. Further, the non-blade portion 82 is provided, in the opening edge, in at least a part other than the region where the electrode 811 functioning as the blade portion is provided. More specifically, the electrode 811 is provided on the suction direction side (the proximal end side Ar2) in the opening edge in the opening section OP. For this reason, by continuously tracing the surface of the tissue piece LT while rotating the tissue piece LT (FIG. 4C and FIG. 5) without moving the electrode 811, it is possible to sequentially scrape off with the electrode 811 and discharge the tissue piece LT to the outside of the body.

That is, unlike the treatment tool described in U.S. Pat. No. 6,024,751 in which the cutting blade moves, in the present embodiment, the electrode 811 functioning as the cutting blade does not move and the tissue piece LT side moves. Therefore, it is possible to scrape off and collect the tissue piece LT. Since the tissue piece LT can freely move on the inside of the bladder BL, the tissue piece LT can rotate with respect to the treatment tool 4. On the other hand, the bladder wall does not move freely. For this reason, for example, even when the distal end portion of the insertion section 8 comes into contact with the bladder wall, the bladder wall is not dug by a fixed depth or more by the electrode 811. Therefore, it is possible to avoid an unintended action on a living tissue. Even when bleeding occurs from the bladder wall, it is also possible to stop the bleeding by pressing the electrode 811 using a publicly-known hemostatic mode by the control device 5.

Since the tissue piece LT is sequentially scraped off and sucked while the tissue piece LT being rotated instead of being finely cut and then sucked, it is possible to improve the collection efficiency of the tissue piece LT.

Therefore, with the treatment tool 4 according to the present embodiment, it is possible to improve the collection efficiency of the tissue piece LT while avoiding an unintended action on the living tissue.

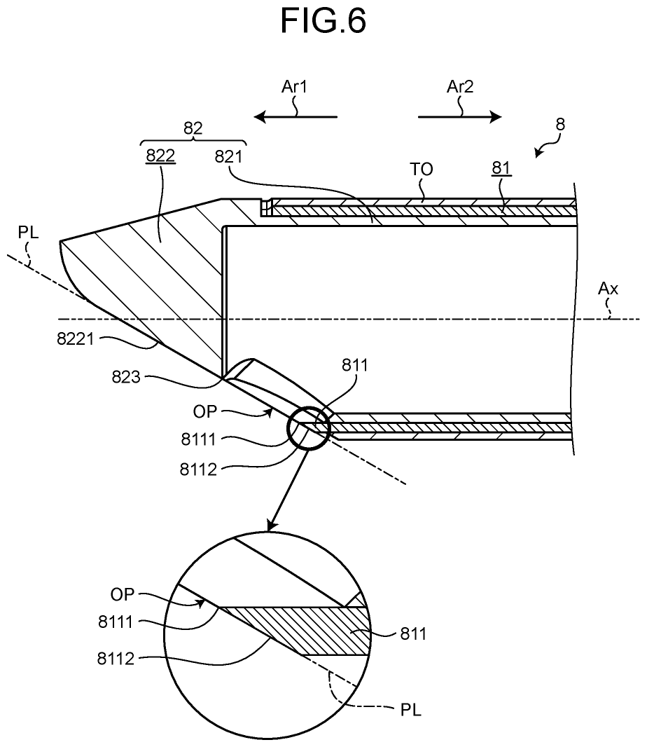

FIG. 6 is a diagram illustrating an effect of the embodiment. Specifically, FIG. 6 is a diagram corresponding to FIG. 3 and illustrates a case where a position where the inclined surface 8112 is provided is a position different from the position illustrated in FIG. 3.

In the electrode 811 illustrated in FIG. 6, the inclined surface 8112 is located on the outer side of the sheath 81 and is provided to extend on the plane PL. That is, in the electrode 811 illustrated in FIG. 6, the edge 8111 and the inclined surface 8112 are located on the plane PL. When the electrode 811 has such a shape, as illustrated in FIG. 6, the tissue piece LT comes into contact with the inclined surface 8112 when the tissue piece LT is sucked from the opening section OP. For this reason, the inclined surface 8112 becomes a physical resistance at the time when the tissue piece LT is sucked from the opening section OP.

In the electrode 811 according to the present embodiment, only the edge 8111 is located on the plane PL. The inclined surface 8112 is located on the inner side of the sheath 81 in the electrode 811 and extends to approach the central axis Ax of the sheath 81 from the edge 8111 toward the proximal end side Ar2.

For this reason, by providing the inclined surface 8112, it is possible to reduce physical resistance at the time when the tissue piece LT is sucked from the opening section OP. Therefore, it is possible to further improve the collection efficiency of the tissue piece LT.

Incidentally, the edge 8111 is a portion having the smallest sectional area in the electrode 811 when the electrode 811 is taken along the plane including the central axis Ax. Such a portion having the smallest sectional area is a portion where electric energy is most concentrated and most contributes to spark discharge when high-frequency power is supplied to between the outer sheath 3 and the sheath 81 and is an important portion for cutting the tissue piece LT.

In the electrode 811 illustrated in FIG. 6, when the tissue piece LT is sucked from the opening section OP, the edge 8111 is covered with the tissue piece LT. Therefore, it is difficult to stabilize the spark discharge at the edge 8111.

On the other hand, in the electrode 811 according to the present embodiment, when the tissue piece LT is sucked from the opening section OP, it is possible to avoid the edge 8111 being covered with the tissue piece LT. That is, it is possible to stabilize the spark discharge at the edge 8111 and further improve the collection efficiency of the tissue piece LT.

The blade portion is the electrode 811 that scrapes the tissue piece LT according to supplied high-frequency power.

Therefore, for example, it is possible to easily burn off, with the electrode 811, even a hard tissue that induces a beach balls (BB) phenomenon and further improve the collection efficiency of the tissue piece LT.

In the treatment tool 4 according to the present embodiment, the non-blade portion 82 configuring a portion other than the electrode 811 in the opening edge of the opening section OP is provided with the sliding surface 8221 that slides the tissue piece LT toward the opening section OP. The sliding surface 8221 and the opening surface of the opening section OP form a continuous surface without a step and are provided on the plane PL intersecting the longitudinal axis of the sheath 81.

For this reason, when the tissue piece LT is scraped off by the electrode 811, the tissue piece LT is not caught by a step. It is possible to smoothly rotate the tissue piece LT and further improve the collection efficiency of the tissue piece LT. Since a part of the non-blade portion 82 made of a material having electric insulation are always positioned on the surface of the tissue piece LT, the tissue piece LT is attracted to the opening section OP without being stuck. Therefore, the tissue piece LT is scraped off while rotating in the opening section OP.

In the treatment tool 4 according to the present embodiment, the non-blade portion 82 is formed by an elastic material and is provided at the distal end of the sheath 81. That is, in the insertion section 8, the distal end that easily comes into contact with the bladder wall or the like is formed by the elastic material.

Therefore, even when the distal end of the insertion section 8 comes into contact with the bladder wall or the like, the bladder wall or the like is not damaged. Therefore, it is possible to satisfactorily avoid an unintended action on a living tissue.

### Other Embodiments

Although the mode for carrying out the disclosure is explained above, the disclosure should not be limited only by the embodiment explained above.

In the embodiment explained above, the electrode 811 is adopted as the blade portion. However, the blade portion is not limited to this and may be a blade or resin that mechanically scrapes a tissue piece. Anything such as a fiber laser may be adopted if the tissue piece can be scraped. In this case, a member such as metal capable of sliding the tissue piece may be adopted as the non-blade portion 82.

In the embodiment explained above, the U-shape is adopted as the shape of the blade portion. However, the shape of the blade portion is not limited to this and other shapes such as a V-shape and an I-shape may be adopted.

### Modification 1

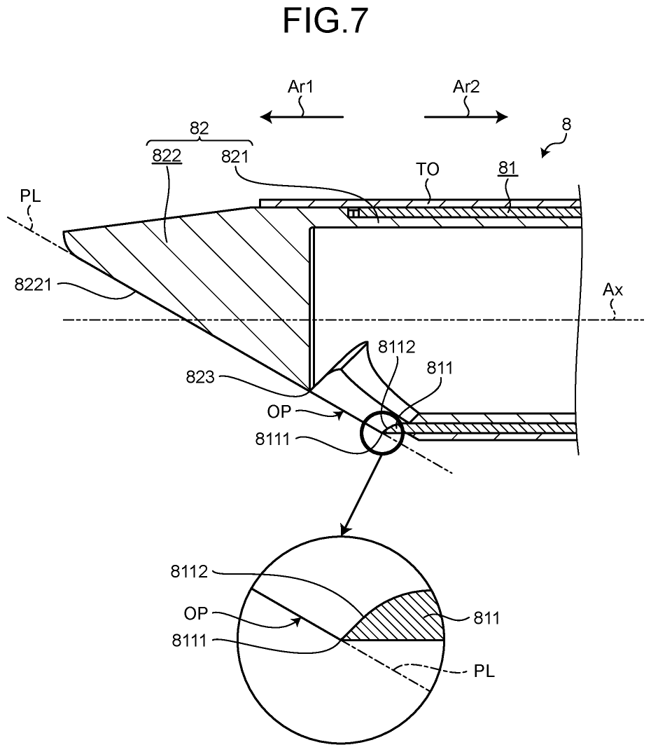

FIG. 7 is a diagram illustrating a modification 1 of the embodiment. Specifically, FIG. 7 is a diagram corresponding to FIG. 3.

In the embodiment explained above, the shape of the inclined surface 8112 may be a shape in this modification 1 illustrated in FIG. 7.

The inclined surface 8112 according to the embodiment explained above has the linearly extending shape when being taken along the plane including the central axis Ax. In contrast, as illustrated in FIG. 7, the inclined surface 8112 according to this modification 1 has a shape extending in a curved shape when being taken along the plane including the central axis Ax. More specifically, the inclined surface 8112 has a shape extending in a curved shape convex to the inner side of the sheath 81 when being taken along the plane including the central axis Ax. Note that the inclined surface 8112 is not limited to the shape extending in the curved shape convex to the inner side of the sheath 81 when being taken along the plane including the central axis Ax. The inclined surface 8112 and may have another shape, for example, a curved shape concave to the outer side of the sheath 81 if the shape is a curved shape.

Even when the inclined surface 8112 according to this modification 1 explained above is adopted, the same effects as the effects of the embodiment explained above are achieved.

### Modification 2

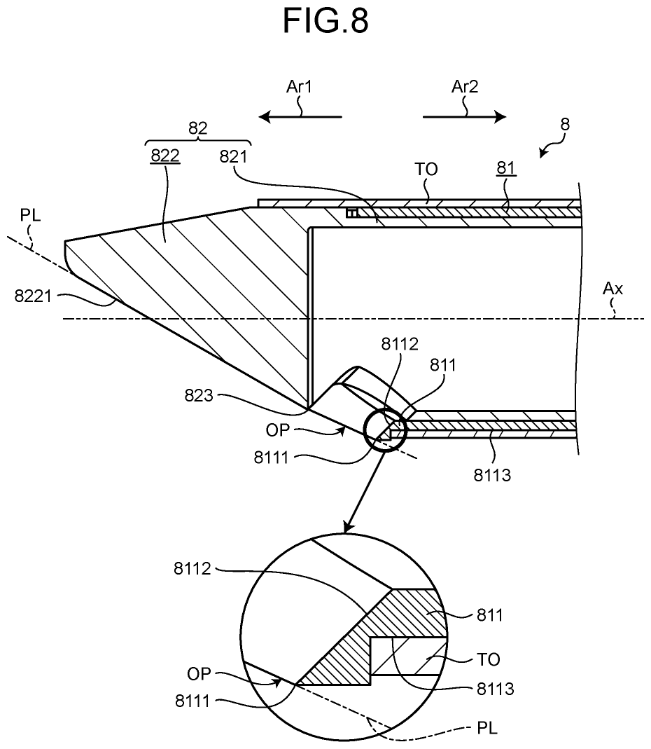

FIG. 8 is a diagram illustrating a modification 2 of the embodiment. Specifically, FIG. 8 is a diagram corresponding to FIG. 3.

In the embodiment explained above, the shape of the electrode 811 may be a shape in this modification 2 illustrated in FIG. 8. Specifically, in the electrode 811 according to this modification 2, as illustrated in FIG. 8, a recess 8113 is provided in the electrode 811 explained in the embodiment explained above.

The recess 8113 is provided on the outer peripheral surface of the electrode 811 (the surface on the outer side of the sheath 81). More specifically, the recess 8113 is a portion recessed from the outer peripheral surface of the electrode 811 toward the inside of the sheath 81 and extends from a position further on the proximal end side Ar2 than the edge 8111 toward the proximal end side Ar2. Here, the depth dimension of the recess 8113 is set to be substantially the same as the thickness dimension of the outer tube TO. A portion on the distal end side Ar1 in the outer tube TO is positioned in the recess 8113. Accordingly, the electrode 811 according to the modification 2 is projected outwardly in the radial direction of the sheath 81 from the outer tube TO.

According to this modification 2 explained above, the following effects are achieved other than the same effects as the effects of the embodiment explained above.

Incidentally, in a state in which the recess 8113 is not provided in the electrode 811 and the outer tube TO is disposed on the outer peripheral surface of the electrode 811 as in the embodiment explained above, the following problem sometimes occurs.

That is, when the tissue piece LT is scraped off by the electrode 811, a problem sometimes occurs in that a part of the tissue piece LT enters between the outer peripheral surface of the electrode 811 and the outer tube TO.

The electrode 811 according to this modification 2 is projected outwardly in the radial direction of the sheath 81 from the outer tube TO. Therefore, when the tissue piece LT is scraped off by the electrode 811, it is possible to avoid the problem explained above occurring and further improve the collection efficiency of the tissue piece LT.

### Modification 3

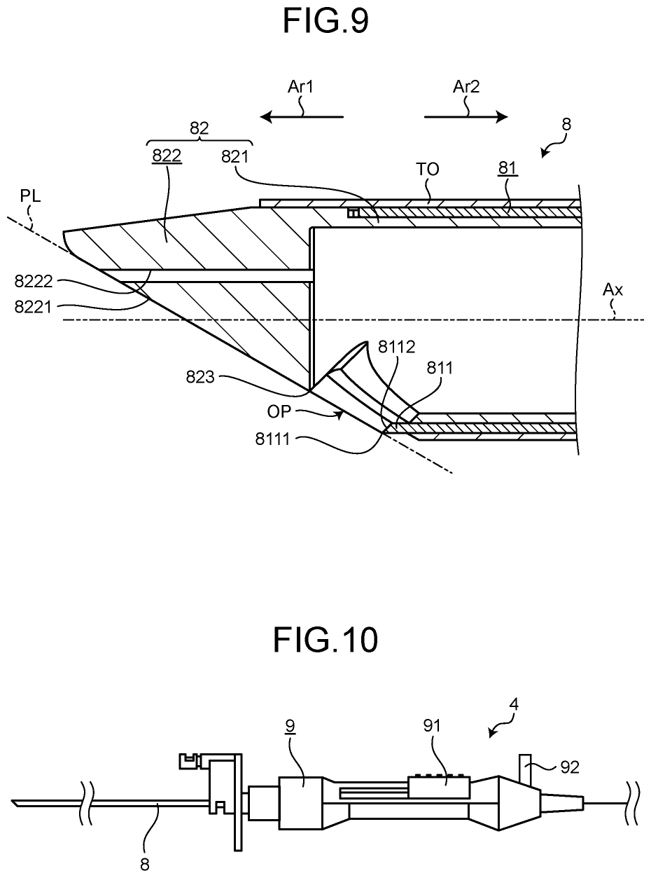

FIG. 9 is a diagram illustrating a modification 3 of the embodiment. Specifically, FIG. 9 is a diagram corresponding to FIG. 3.

In the embodiment explained above, the shape of the non-blade portion main body 822 may be a shape in this modification 3 illustrated in FIG. 9. Specifically, in the non-blade portion main body 822 according to this modification 3, as illustrated in FIG. 9, the non-blade portion main body 822 explained in the embodiment explained above is provided with a through-hole 8222 that penetrates the non-blade portion main body 822, from the sliding surface 8221 toward the proximal end side Ar2, to the inside of the insertion section 821 and communicates between the inside and the outside of the sheath 81.

According to the modification 3 explained above, the following effects are achieved besides the same effects as the effects of the embodiment explained above.

When the through-hole 8222 is not provided in the non-blade portion main body 822 as in the embodiment explained above, the following problem sometimes occurs.

That is, when the tissue piece LT is scraped off by the electrode 811, the opening section OP is sometimes closed by the tissue piece LT. In this case, a problem sometimes occurs in that perfusion of the physiological saline tracing a path from the water feeding port 31 to the outer sheath 3, the hole for water feeding 32, the opening section OP, the sheath 81, and the suction device 7 is delayed.

A through-hole 8222 is provided in the non-blade portion main body 822 according to this modification 3. For this reason, even when the opening section OP is closed by the tissue piece LT when the tissue piece LT is scraped off by the electrode 811, it is possible to perfuse the physiological saline to trace a route from the water feeding port 31 to the outer sheath 3, the hole for water feeding 32, the through-hole 8222, the sheath 81, and the suction device 7. It is possible to avoid the temperature of the inside of the bladder BL becoming unnecessarily high.

### Modification 4

FIG. 10 is a diagram for explaining a modification 4 of the embodiment. Specifically, FIG. 10 is a diagram illustrating a treatment tool 4 according to the modification 4.

In the embodiment explained above, a valve 92 may be provided in the housing 9 as in this modification 4 illustrated in FIG. 10.

The valve 92 is a valve that enables a suction conduit (not illustrated) on the inside of the housing 9 and the outside of the housing 9 to communicate. The valve 92 is opened when the pressure of the suction conduit falls below a predetermined negative pressure to cause the suction conduit with the outside of the housing 9 to communicate.

According to this modification 4 explained above, the following effects are achieved besides the same effects as the effects of the embodiment explained above.

Incidentally, as the suction device 7, for example, it is conceivable to use a suction device regularly provided in a hospital. In this case, the suction pressure of the tissue piece LT from the opening section OP varies depending on a suction device in use. That is, the collection efficiency of the tissue piece LT fluctuates depending on the suction device in use.

In the modification 4, the valve 92 is provided in the housing 9. Therefore, even when a suction device having any performance is used, it is possible to fix the suction pressure of the tissue piece LT from the opening section OP. It is possible to stabilize the collection efficiency of the tissue piece LT.

### Modification 5

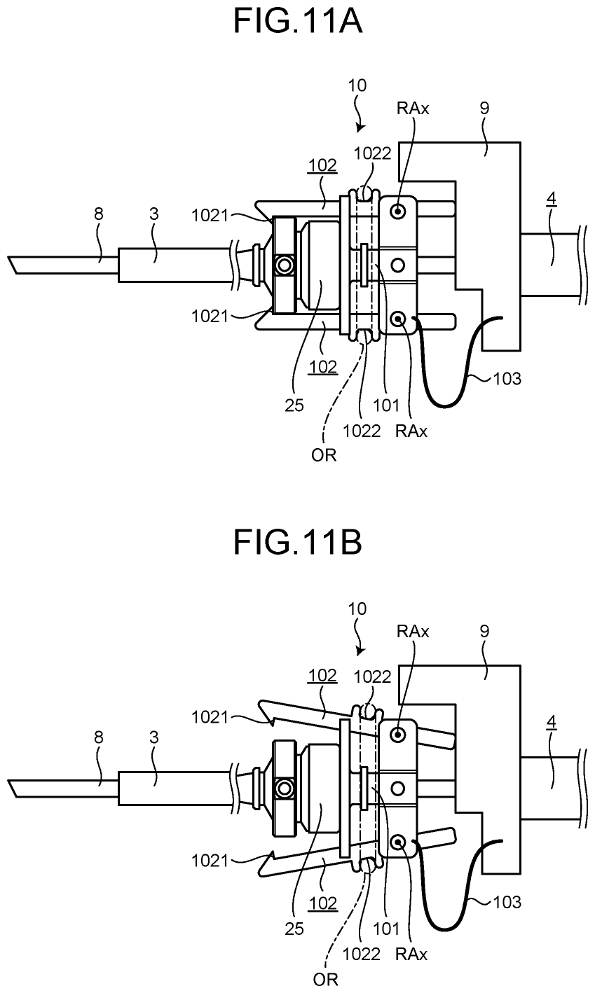

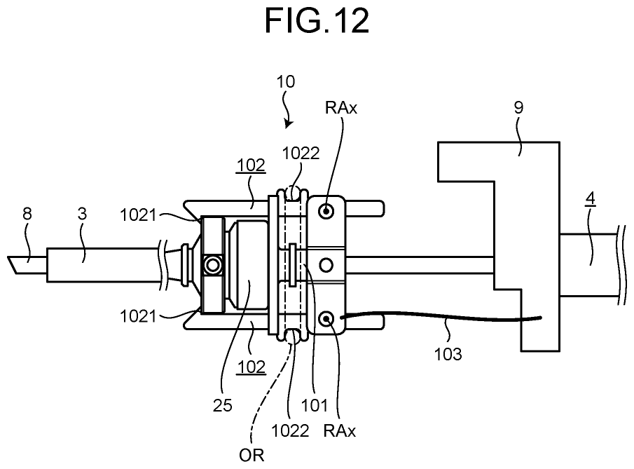

FIGS. 11A and 11B and FIG. 12 are diagrams for explaining a modification 5 of the embodiment.

In the embodiment explained above, a lock device 10 according to this modification 5 illustrated in FIGS. 11A and 11B and FIG. 12 may be provided.

As illustrated in FIGS. 11A and 11B and FIG. 12, the lock device 10 includes a support section 101, a pair of lock sections 102, and a wire 103.

The support section 101 is a portion that supports the pair of lock sections 102 and the wire 103.

The pair of lock sections 102 is disposed in a vertically facing state In FIGS. 11A and 11B and FIG. 12 with the support section 101 interposed therebetween. Note that the pair of lock sections 102 has a vertically symmetrical shape in FIGS. 11A and 11B and FIG. 12. For this reason, one lock section 102 is explained below.

As illustrated in FIGS. 11A and 11B and FIG. 12, the lock section 102 includes a claw section 1021 on the distal end side Ar1 and is supported to be capable of turning with respect to the support section 101 centering on a turning axis RAx extending in a direction orthogonal to the paper surface of FIGS. 11A and 11B and FIG. 12.

Here, on the outer surface of the lock section 102, as illustrated in FIGS. 11A and 11B and FIG. 12, a recess 1022 to which an O-ring OR is locked is provided at a position between the turning axis RAx and the claw section 1021. The O-ring OR is locked to recesses 1022 of the pair of lock sections 102 in a state in which the support section 101 and the pair of lock sections 102 are inserted through the inside of the O-ring OR. Note that, in FIGS. 11A and 11B and FIG. 12, for convenience of explanation, the O-ring OR is indicated by an alternate long and short dash line.

One end of the wire 103 is fixed to the support section 101 and the other end is fixed to the housing 9.

The lock device 10 is fixed to the housing 9 via the wire 103. As illustrated in FIGS. 11A and 11B and FIG. 12, a pair of claw sections 1021 us locked to the proximal end portion 25 of the endoscope 2, whereby the lock device 10 restricts the movement of the treatment tool 4 to the right side in FIGS. 11A and 11B and FIG. 12. When a locked state of the pair of claw sections 1021 and the proximal end portion 25 is released, as illustrated in FIG. 11B, the pair of lock sections 102 only has to be turned with respect to the support section 101 centering on the turning axis RAx against an urging force of the O-ring OR.

A method of using the lock device 10 is as follows.

First, a user inserts the insertion section 8 through the support section 101 and fixes the lock device 10 to the housing 9 via the wire 103. Subsequently, the user inserts the insertion section 8 through the first and second through-holes of the endoscope 2. At this time, the claw sections 1021 collide with the proximal end portion 25 and the pair of lock sections 102 turns in a direction in which the pair of lock sections 102 opens against the urging force of the O-ring OR (FIG. 11B). When the claw section 1021 moves beyond the proximal end portion 25, the pair of lock sections 102 returns to the original positions with the urging force of the O-ring OR (FIG. 11A). Then, the pair of claw sections 1021 is locked to the proximal end portion 25, whereby the movement of the lock device 10 to the right side in FIGS. 11A and 11B and FIG. 12 is restricted. In this state, when the user tries to pull out the insertion section 8 from the first and second through-holes, the pulling out is restricted by the wire 103 as illustrated in FIG. 12. More specifically, a minimum separation distance between the distal end portion (the electrode 811) of the insertion section 8 and the distal end (the distal end portion 22) of the outer sheath 3 is limited by the length of the wire 103.

Note that, in the above explanation, the O-ring OR is used as means for returning the pair of lock sections 102 to the original positions. However, the means is not limited to this. Another elastic material such as a leaf spring made of metal may be adopted.

According to the modification 5 explained above, the following effects are achieved besides the same effects as the effects of the embodiment explained above.

Incidentally, it is assumed that, in a state in which high-frequency power is supplied to between the outer sheath 3 and the sheath 81, the user tries to pull out the insertion section 8 of the treatment tool 4 from the first and second through-holes in the endoscope 2 and the electrode 811 and the distal end portion 22 approach. In this case, there is a problem occurs in that electric discharge occurs between the electrode 811 and the distal end portion 22 and the distal end portion 22 is deteriorated.

In this modification 5, the minimum separation distance between the electrode 811 and the distal end portion 22 is limited by the lock device 10 (the wire 103). For this reason, the problem explained above can be avoided.

### Modification 6

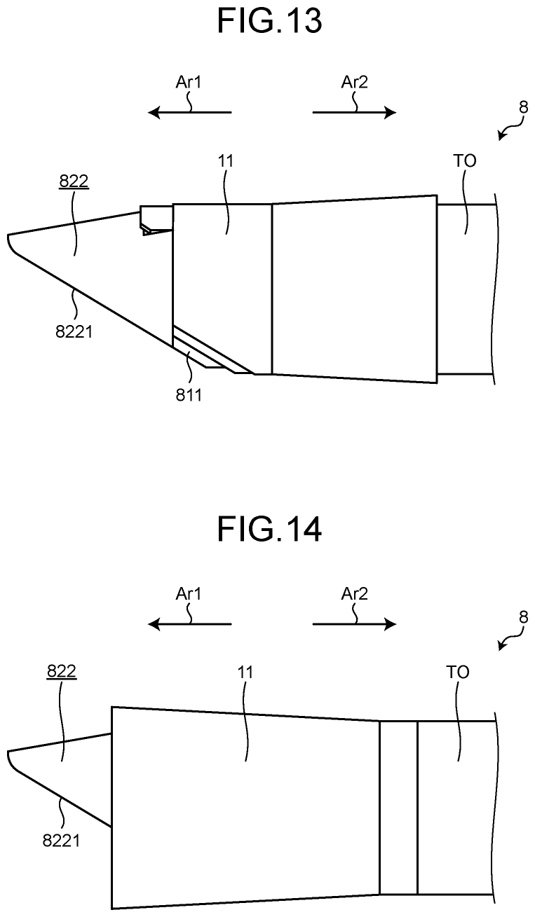

FIG. 13 and FIG. 14 are diagrams for explaining a modification 6 of the embodiment.

In the embodiment explained above, an insulating cover 11 according to this modification 6 illustrated in FIG. 13 and FIG. 14 may be provided.

The insulating cover 11 is made of rubber or the like having electrical insulation and elasticity and has a cylindrical shape. As illustrated in FIG. 13, in a state in which an end portion on the distal end side Ar1 in the insertion section 8 is inserted, the insulating cover 11 is connected to the end portion. In this state, the electrode 811 is not covered by the insulating cover 11 and is exposed to the outside. Then, when the user tries to pull out the insertion section 8 from the first and second through-holes in the endoscope 2, the end face on the proximal end side Ar2 of the insulating cover 11 is caught by the edge of the first through-hole and, as illustrated in FIG. 14, a part on the proximal end side Ar2 is turned up toward the distal end side Ar1 to cover the electrode 811.

According to this modification 6 explained above, the following effects are achieved besides the same effects as the effects of the embodiment explained above.

Incidentally, it is assumed that, in a state in which high-frequency power is supplied to between the outer sheath 3 and the sheath 81, the user tries to pull out the insertion section 8 of the treatment tool 4 from the first and second through-holes in the endoscope 2 and the electrode 811 and the distal end portion 22 approach. In this case, there is a problem occurs in that electric discharge occurs between the electrode 811 and the distal end portion 22 and the distal end portion 22 is deteriorated.

In this modification 6, a configuration in which the electrode 811 is covered with the insulating cover 11 when the insertion section 8 is pulled out from the first and second through-holes of the endoscope 2 is adopted. For this reason, the problem explained above can be avoided.

With the treatment tool, the treatment system, and the method of operating the treatment system according to the disclosure, it is possible to improve the collection efficiency of a tissue piece while avoiding unintended action on the living tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool, comprising:
a tubular portion including a tubular portion main body having a long tubular shape and an opening section provided in the tubular portion main body, the tubular portion being configured to suck a tissue piece from the opening section;
a blade portion provided in a part of an opening edge in the opening section; and
a non-blade portion provided in at least a part other than a region where the blade portion is provided in the opening edge, the non-blade portion being configured to slide the tissue piece,
wherein the blade portion includes an edge having a smallest sectional area in a cross section of the blade portion taken along a plane including a central axis of the tubular portion main body, and only the edge is located on an imaginary plane obtained by extending an opening surface of the opening section.

2. The treatment tool according to claim 1, wherein the blade portion further includes an inclined surface located on an inner side of the tubular portion and connected to the edge, and
wherein the inclined surface extends from the edge such that a proximal end side of the inclined surface approaches the central axis of the tubular portion main body.

3. The treatment tool according to claim 1, wherein the blade portion is provided on a proximal end side at the opening edge, and
wherein the non-blade portion is provided on a distal end side at the opening edge.

4. The treatment tool according to claim 2, wherein the inclined surface is a surface intersecting the opening surface of the opening section.

5. The treatment tool according to claim 4, wherein the inclined surface is a surface extending in a normal direction of the opening surface of the opening section.

6. The treatment tool according to claim 1, wherein the blade portion is an electrode capable of scraping the tissue piece according to supplied high-frequency power.

7. The treatment tool according to claim 1, further comprising an insulating portion having electric insulation and covering an outer peripheral surface of the tubular portion main body,
wherein the blade portion has a shape projecting outwardly in a radial direction of the tubular portion main body from the insulating portion.

8. The treatment tool according to claim 1, wherein the non-blade portion is provided with a through-hole communicating between an inside and an outside of the tubular portion.

9. A treatment system, comprising:
a treatment tool configured to be inserted into a subject; and
a suction device configured to suck a tissue piece through the treatment tool,
wherein the treatment tool includes:
a tubular portion including a tubular portion main body having a long tubular shape and an opening section provided in the tubular portion main body, the tubular portion being configured to suck the tissue piece from the opening section;
a blade portion provided in a part of an opening edge in the opening section; and
a non-blade portion provided in at least a part other than a region where the blade portion is provided in the opening edge, the non-blade portion being configured to slide the tissue piece, and
wherein the blade portion includes an edge having a smallest sectional area in a cross section of the blade portion taken along a plane including a central axis of the tubular portion main body, and only the edge is located on an imaginary plane obtained by extending an opening surface of the opening section.

10. The treatment system according to claim 9, wherein the blade portion further includes an inclined surface located on an inner side of the tubular portion and connected to the edge, and
wherein the inclined surface extends from the edge such that a proximal end side of the inclined surface approaches the central axis of the tubular portion main body.

11. The treatment system according to claim 9, wherein the blade portion is provided on a proximal end side at the opening edge, and
wherein the non-blade portion is provided on a distal end side at the opening edge.

12. The treatment system according to claim 9, wherein the blade portion is an electrode capable of scraping the tissue piece according to supplied high-frequency power.

13. The treatment system according to claim 9, further comprising an insulating portion having electric insulation and covering an outer peripheral surface of the tubular portion main body,
wherein the blade portion has a shape projecting outwardly in a radial direction of the tubular portion main body from the insulating portion.

14. The treatment system according to claim 9, wherein the non-blade portion is provided with a through-hole communicating between an inside and an outside of the tubular portion.

15. A method of operating a treatment system including a treatment tool configured to be inserted into a subject and a suction device configured to suck a tissue piece through the treatment tool,
the treatment tool including a tubular portion including a tubular portion main body having a long tubular shape and an opening section provided in the tubular portion main body, the tubular portion being configured to suck the tissue piece from the opening section, a blade portion provided in a part of an opening edge in the opening section, and a non-blade portion provided in at least a part other than a region where the blade portion is provided in the opening edge, the non-blade portion being configured to slide the tissue piece,
the blade portion includes an edge having a smallest sectional area in a cross section of the blade portion taken along a plane including a central axis of the tubular portion main body, and only the edge is located on an imaginary plane obtained by extending an opening surface of the opening section,
the method comprising:
scraping the tissue piece with the blade portion by applying high-frequency power to the blade portion while attracting the tissue piece to the opening section of the tubular portion main body with suction of the suction device; and
collecting the scraped tissue piece from the tubular portion main body with the suction.

16. The method of operating the treatment system according to claim 15, wherein the blade portion further includes an inclined surface located on an inner side of the tubular portion and connected to the edge, and
wherein the inclined surface extends from the edge such that a proximal end side of the inclined surface approaches the central axis of the tubular portion main body.

17. The method of operating the treatment system according to claim 15, wherein the blade portion is provided on a proximal end side at the opening edge, and
wherein the non-blade portion is provided on a distal end side at the opening edge.

18. The method of operating the treatment system according to claim 15, wherein the blade portion is an electrode capable of scraping the tissue piece according to supplied high-frequency power.

19. The method of operating the treatment system according to claim 15, wherein the treatment system further includes an insulating portion having electric insulation and covering an outer peripheral surface of the tubular portion main body, and the blade portion has a shape projecting outwardly in a radial direction of the tubular portion main body from the insulating portion.

20. The method of operating the treatment system according to claim 15, wherein the non-blade portion is provided with a through-hole communicating between an inside and an outside of the tubular portion.
